(19) United States
(12) Patent Application Publication
(10) Pub. No.: US 2026/0245692 A1
(43) Pub. Date: Aug. 20, 2026

(54) EMERGENCY INCIDENT REHABILITATION SYSTEM AND METHOD

(71) Applicant: James Nicholas McManus, Lebanon, NJ (US)

(72) Inventor: James Nicholas McManus, Lebanon, NJ (US)

(21) Appl. No.: 19/542,156

(22) Filed: Feb. 17, 2026

Related U.S. Application Data
(60) Provisional application No. 63/759,845, filed on Feb. 18, 2025.

(51) Int. Cl. G16H 20/00; G16H 10/60; G16H 40/20; G16H 40/67; G16H 70/20

(52) U.S. Cl. CPC G16H 20/00; G16H 10/60; G16H 40/20; G16H 40/67; G16H 70/20

(57) ABSTRACT

An emergency incident rehabilitation system and method which enables consistent, high-quality rehabilitation for various types of first responders with minimal training requirements. The system comprises role-specific instruction cards worn on lanyards, accountability tracking forms, individual tracker forms for medical documentation, rehabilitation area layout guides, and medical screening checklists. The method includes distributing role cards to personnel, establishing a rehabilitation area, tracking first responders through check-in and check-out procedures, conducting initial and exit medical screenings, monitoring vital signs at regular intervals, and determining appropriate disposition based on medical evaluation. The system is scalable, modular, and adaptable to varying incident conditions and different types of first responders, including firefighters, emergency medical services personnel, law enforcement officers, search and rescue teams, hazardous materials specialists, and other emergency workers.

### CROSS-REFERENCE TO RELATED APPLICATION

This application claims the benefit of priority to U.S. Provisional Patent Application Serial No. 63/759,845, filed February 18, 2025, entitled "FIREFIGHTER REHABILITATION SYSTEM AND METHOD," the entire contents of which are incorporated herein by reference.

### FIELD OF THE INVENTION

The present invention relates generally to emergency medical services and incident management systems. More particularly, the invention relates to systems and methods for managing emergency incident rehabilitation for first responders at emergency incident scenes, providing accountability, medical evaluation, rest, and replenishment for emergency personnel during emergency operations, including but not limited to firefighters, emergency medical services personnel, law enforcement officers, search and rescue teams, and other emergency responders.

### BACKGROUND OF THE INVENTION

First responders face significant health risks during emergency operations, including cardiac events resulting from overexertion, heat stress, dehydration, and physical exhaustion. For firefighters specifically, cardiac events from overexertion and stress represent the leading cause of line-of-duty fatalities. Similarly, law enforcement personnel, emergency medical technicians, search and rescue teams, and other emergency workers experience elevated health risks during extended or intense emergency operations. Many of these adverse health events are preventable through effective emergency incident rehabilitation.

Emergency incident rehabilitation serves four primary functions: accountability (tracking personnel in and out of designated areas), rest (providing relief from environmental conditions and operational duties), medical evaluation and treatment, and replenishment (providing fluids and nutrients). Despite the critical importance of emergency incident rehabilitation, personnel who staff these rehabilitation areas are often not formally trained in this function and perform it infrequently. When emergency incident rehabilitation is performed, it frequently lacks standardization and effectiveness, thereby putting lives at risk.

The quality and consistency of rehabilitation efforts vary significantly from incident to incident and from agency to agency. Different types of emergency incidents present unique rehabilitation challenges. For example, firefighting operations involve heat stress and smoke exposure; hazardous materials incidents involve chemical exposure concerns; search and rescue operations involve physical exhaustion over extended periods; and mass casualty incidents require management of large numbers of responders simultaneously.

Conventional systems for emergency incident rehabilitation are complex, impractical for field deployment, and expensive. Professional organizations have issued guidance that is often aspirational, conflicting, and unrealistic for real-world implementation. These systems typically require extensive training and large numbers of well-trained personnel, making them unsuitable for the varying conditions encountered at emergency incidents, including different types of incidents, varying durations, different times of day, and diverse weather conditions.

There exists a need in the art for an emergency incident rehabilitation system that is simple, flexible, modular, and sustainable. There is a particular need for a system that can be effectively implemented by personnel with minimal training, that provides clear role-specific instructions, and that can be scaled up or down depending on the size and nature of the incident. There is also a need for a system that maintains consistent accountability of all first responders while ensuring timely medical evaluation and treatment, regardless of the type of emergency responder being rehabilitated.

### SUMMARY OF THE INVENTION

The present invention addresses the deficiencies of prior art systems by providing a comprehensive emergency incident rehabilitation system and method that enables agencies to conduct consistently high-quality emergency incident rehabilitation for all types of first responders, regardless of the training level of personnel staffing the rehabilitation areas, the type or duration of the incident, the time of day, weather conditions, or other variables.

In one aspect, the present invention provides an emergency incident rehabilitation system comprising a plurality of role-specific instruction cards configured to be worn on lanyards by personnel, wherein each role card includes written instructions specifying the responsibilities, required equipment, required certifications, reporting relationships, and designated location for that role. The system further comprises a rehabilitation area layout guide, a plurality of accountability tracking forms, individual patient care record forms, and medical screening checklists.

In another aspect, the present invention provides a method for managing emergency incident rehabilitation at an emergency incident scene, comprising: selecting a rehabilitation manager; distributing role-specific instruction cards to personnel; establishing a rehabilitation area according to a standardized layout guide; tracking first responders entering and exiting the rehabilitation area using accountability forms; conducting initial medical screenings; monitoring vital signs at regular intervals; recording medical data on individual tracker forms; evaluating first responders for release to duty or transport to medical facilities; and maintaining continuous accountability of all personnel.

The system is designed to be "personnel-proof" in that it is sufficiently simple, flexible, modular, and sustainable to be effectively implemented by any number of staff members with minimal training. Each role is clearly explained on a lanyard-mounted card, enabling personnel to immediately understand and execute their assigned responsibilities. The system provides a framework within which trained medical personnel can effectively communicate and exercise their medical judgment while ensuring that basic accountability and evaluation protocols are consistently maintained.

The system is scalable, allowing for the creation of additional rehabilitation sections as needed and the reduction of sections when appropriate. The system is also portable, permitting the rehabilitation area to be moved either gradually as responders complete their rest periods or rapidly in emergency situations. The system is adaptable to various types of emergency incidents and various types of first responders, including firefighters, emergency medical services personnel, law enforcement officers, hazardous materials teams, search and rescue personnel, and other emergency workers.

These and other features, aspects, and advantages of the present invention will become better understood with reference to the following drawings, description, and claims.

### BRIEF DESCRIPTION OF THE DRAWINGS

FIG. 1 depicts an Individual Tracker form for use in accordance with a preferred embodiment of the present invention;

FIG. 2 depicts a Manager Accountability Record form for use in accordance with a preferred embodiment of the present invention;

FIG. 3 depicts a Rehabilitation Map form for use in accordance with a preferred embodiment of the present invention;

FIG. 4 depicts Rehabilitation Placement recommendations for use in accordance with a preferred embodiment of the present invention;

FIG. 5 depicts Rehabilitation Check Out Requirements for use in accordance with a preferred embodiment of the present invention;

FIG. 6 depicts the reverse of an exemplary Recorder role card for use in accordance with a preferred embodiment of the present invention;

FIG. 7 depicts the reverse of an exemplary Evaluator role card for use in accordance with a preferred embodiment of the present invention;

FIG. 8 depicts the front of an exemplary Recorder role card for use in accordance with a preferred embodiment of the present invention;

FIG. 9 depicts the rear of an exemplary Rehabilitation Manager role card for use in accordance with a preferred embodiment of the present invention;

FIG. 10 depicts the front of an exemplary Evaluator role card for use in accordance with a preferred embodiment of the present invention;

FIG. 11 depicts the front of an exemplary Rehabilitation Manager role card for use in accordance with a preferred embodiment of the present invention;

FIG. 12 depicts a flowchart showing the initial setup and assignment process of the emergency incident rehabilitation system in accordance with a preferred embodiment of the present invention; and

FIG. 13 depicts a flowchart showing the medical evaluation, monitoring, and disposition process of the emergency incident rehabilitation system in accordance with a preferred embodiment of the present invention.

### DETAILED DESCRIPTION OF THE INVENTION

The following detailed description represents the best currently contemplated modes of carrying out exemplary embodiments of the invention. The description is not to be taken in a limiting sense but is made for the purpose of illustrating the general principles of the invention, since the scope of the invention is best defined by the appended claims.

As described above, first responders face significant health risks during emergency operations. For firefighters, cardiac events from overexertion and stress represent the leading cause of line-of-duty fatalities. Other emergency personnel, including emergency medical technicians, law enforcement officers, search and rescue teams, hazardous materials specialists, and other emergency workers, similarly face health risks from heat stress, dehydration, physical exhaustion, and overexertion during emergency operations. Many of these adverse health events are preventable through high-quality emergency incident rehabilitation.

However, personnel who typically staff rehabilitation areas, often emergency medical technicians or paramedics, are frequently not formally trained in emergency incident rehabilitation and perform this function infrequently. Consequently, emergency incident rehabilitation quality and consistency vary greatly from incident to incident and from agency to agency. The present invention solves these and other problems associated with conventional systems and methods by providing a comprehensive, standardized approach applicable to all types of first responders.

### System Components and Organization

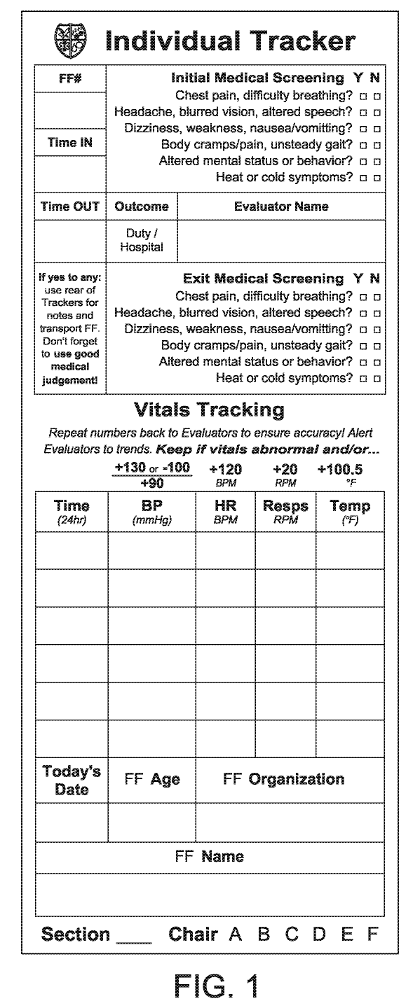

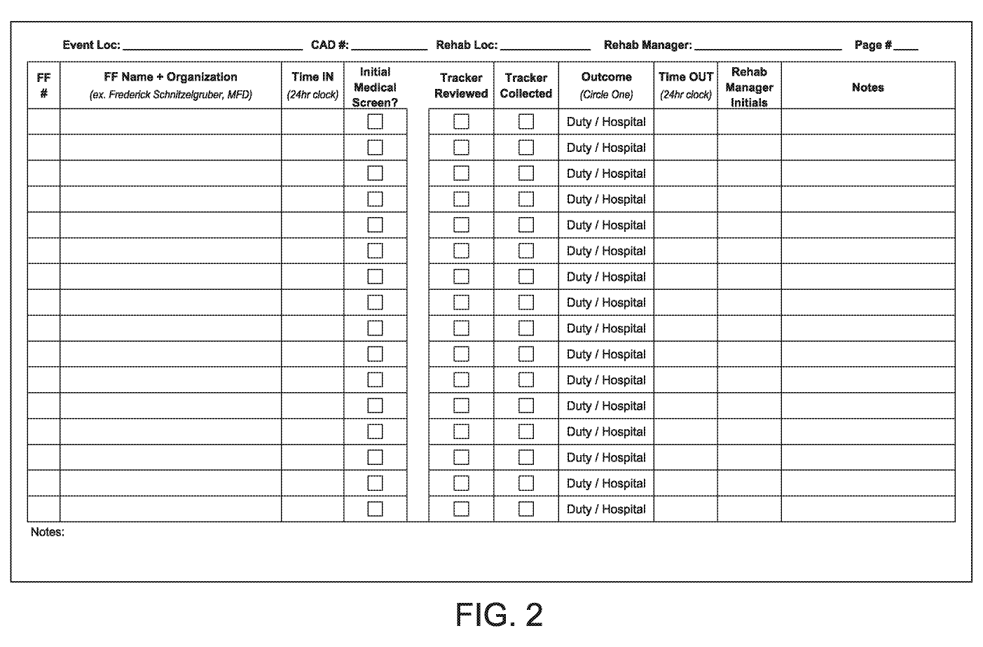

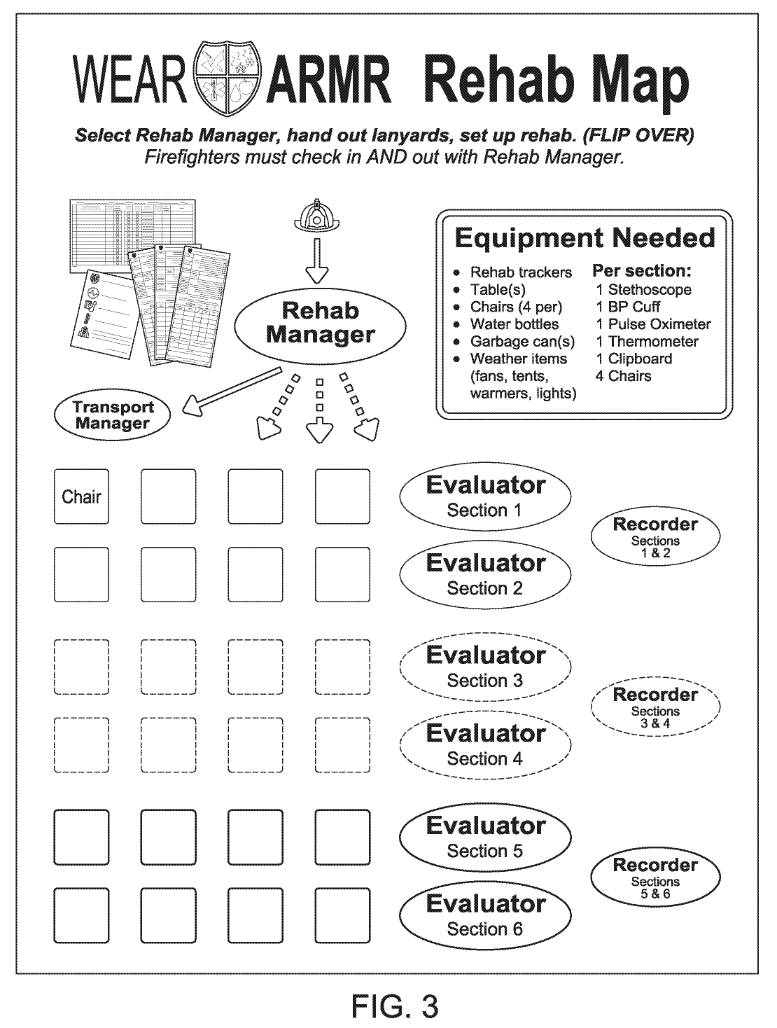

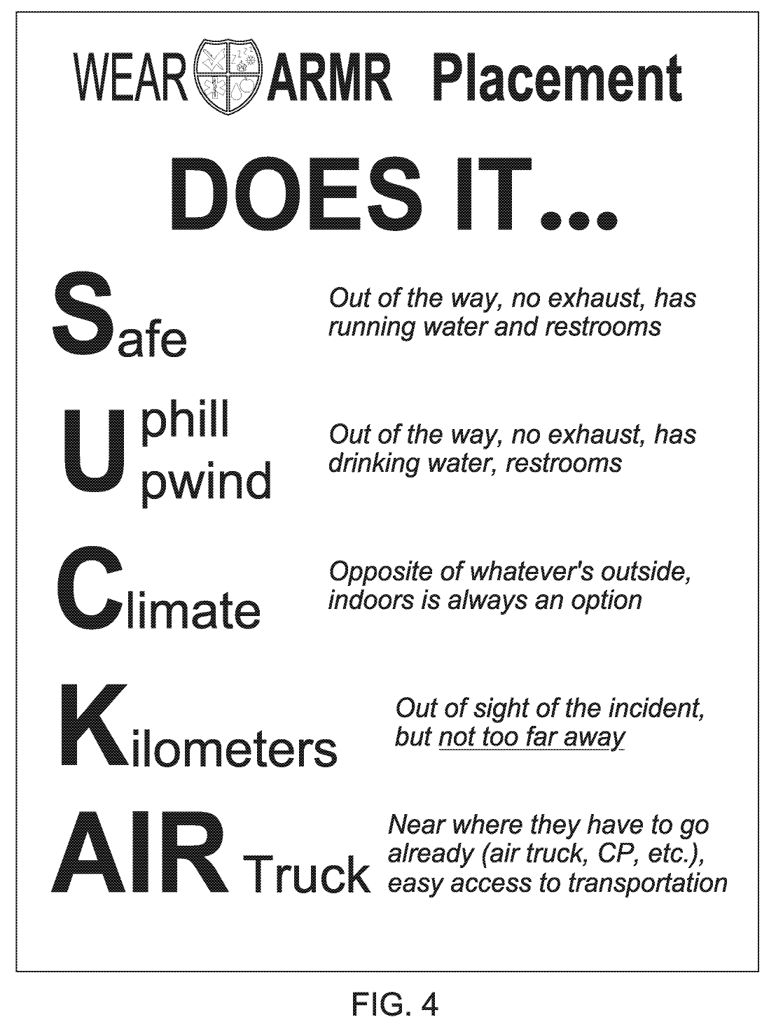

Referring now to FIGS. 1-13, the present invention comprises several integrated components that work together to provide comprehensive emergency incident rehabilitation for various types of first responders. The primary components include: role-specific instruction cards configured to be worn on lanyards; a rehabilitation manager clipboard containing organizational materials; accountability tracking forms including the Manager Accountability Record (FIG. 2); Individual Tracker forms (FIG. 1) for recording medical evaluations; a Rehabilitation Map (FIG. 3) providing layout guidance; and Check Out Requirements documentation (FIG. 4) for ensuring proper release procedures.

While the system is described herein with examples related to firefighting operations for illustrative purposes, it should be understood that the system is equally applicable to other types of emergency incidents and other types of first responders. The term "first responder" as used herein encompasses firefighters, emergency medical services personnel (including emergency medical technicians and paramedics), law enforcement officers, search and rescue team members, hazardous materials specialists, disaster response personnel, and any other emergency workers who may require rehabilitation during emergency operations.

### Medical Evaluation Process

Referring to FIG. 1, the Individual Tracker form serves as a comprehensive patient care record for each first responder undergoing rehabilitation. The reverse of the form is deliberately left blank to allow for miscellaneous note-taking (of specific symptoms, narratives, and/or additional vital sign tracking) that may be necessary. Upon arrival in their assigned section, responders are seated and, if not already accomplished in accordance with standard operating guidelines and safety protocols, they remove protective equipment or gear as appropriate for their role and the type of incident.

The Recorder for each section maintains two small stacks of blank Individual Trackers on a clipboard, one stack for each section to which they are assigned. For each arriving first responder, the Recorder records identifying information including name, age, agency or unit, section number, and seat letter on an Individual Tracker form.

The Evaluator then administers the Initial Medical Screening questionnaire, which appears at the top portion of the Individual Tracker form. This questionnaire assesses subjective signs, symptoms, and complaints that may vary depending on the type of emergency incident. The questions are designed to assist responders in identifying a wide range of potential medical issues that may be encountered across first responder communities (EMS, firefighting, search and rescue, hazardous materials, etc.), including heart attacks, temperature-induced impairments or emergencies, smoke inhalation, chemical exposure, and more. Symptoms are grouped together to help personnel administer the questionnaire more quickly, while also checking for symptoms often found together and indicative of potentially similar underlying issues. For example, body cramps/pain are listed with unsteady gait, as both suggest fine and/or gross motor impairment.

Following the Initial Medical Screening, the Evaluator evaluates the first responder's vital signs, including blood pressure, heart rate, body temperature, and blood oxygen saturation (SPO2). These vital signs provide objective measures of the responder's physiological condition and are applicable across all types of emergency responders.

The Evaluator communicates the vital signs measurements to the Recorder, who documents the information on the Individual Tracker form. Both the Recorder and the Evaluator then compare the measured vital signs against a "Keep If" checklist, which provides threshold values above each column on the Individual Tracker form. These threshold values indicate when vital signs are outside acceptable ranges, requiring continued monitoring or medical intervention.

The Evaluator continues to assess other first responders assigned to their section (typically between four and six individuals) and repeats vital sign evaluations at intervals of approximately five to ten minutes. This regular monitoring allows for the detection of trends, either improvement or deterioration, in the responder's condition. The Evaluator passes updated vital signs information to the Recorder for each successive evaluation cycle.

### Accountability Management

Accountability is maintained through multiple integrated mechanisms. As shown in FIG. 2, the Manager Accountability Record provides a centralized tracking system for all first responders entering and exiting the rehabilitation area. When first responders arrive at the rehabilitation area, the Rehabilitation Manager employs one of two accountability methods depending on whether the responders possess accountability tags.

If arriving first responders have an accountability tag, the Rehabilitation Manager collects and clips the tag to an Accountability Tag Board, then assigns the responder to a specific rehabilitation section. The Rehabilitation Manager records the responder demographic information on the left side of the Manager Accountability Record, including name, ID number, agency or unit, and section assignment.

If arriving first responders do not possess an accountability tag, the Rehabilitation Manager writes the responder demographic information directly on the left side of the Manager Accountability Record, then assigns the responder to a rehabilitation section. This dual approach ensures accountability regardless of whether an agency utilizes a formal accountability tag system, and is adaptable to the accountability practices of different emergency services agencies.

### Medical Decision Making and Disposition

The Evaluator exercises medical judgment throughout the rehabilitation process to determine appropriate disposition for each first responder. If a responder's vital signs normalize and no concerning signs or symptoms are present, the Evaluator conducts an Exit Medical Screening using the questionnaire provided at the top of the Individual Tracker form. Upon satisfactory completion of the Exit Medical Screening, the responder and the completed Individual Tracker are sent back to the Rehabilitation Manager for the checkout process.

If a first responder's vital signs deteriorate over time, do not normalize, or if concerning signs and symptoms emerge during the Exit Medical Screening, the Evaluator exercises medical judgment to determine whether the responder should be transported to a hospital or whether advanced life support (ALS) services should be engaged.

When a decision is made to transport a first responder, the Transport Manager arranges for a standby ambulance and crew to transport the responder to an appropriate medical facility. The Transport Manager ensures that correct documentation, including the completed Individual Tracker form, accompanies the responder. The Transport Manager then communicates this information to the Rehabilitation Manager for accountability purposes.

### Checkout Process

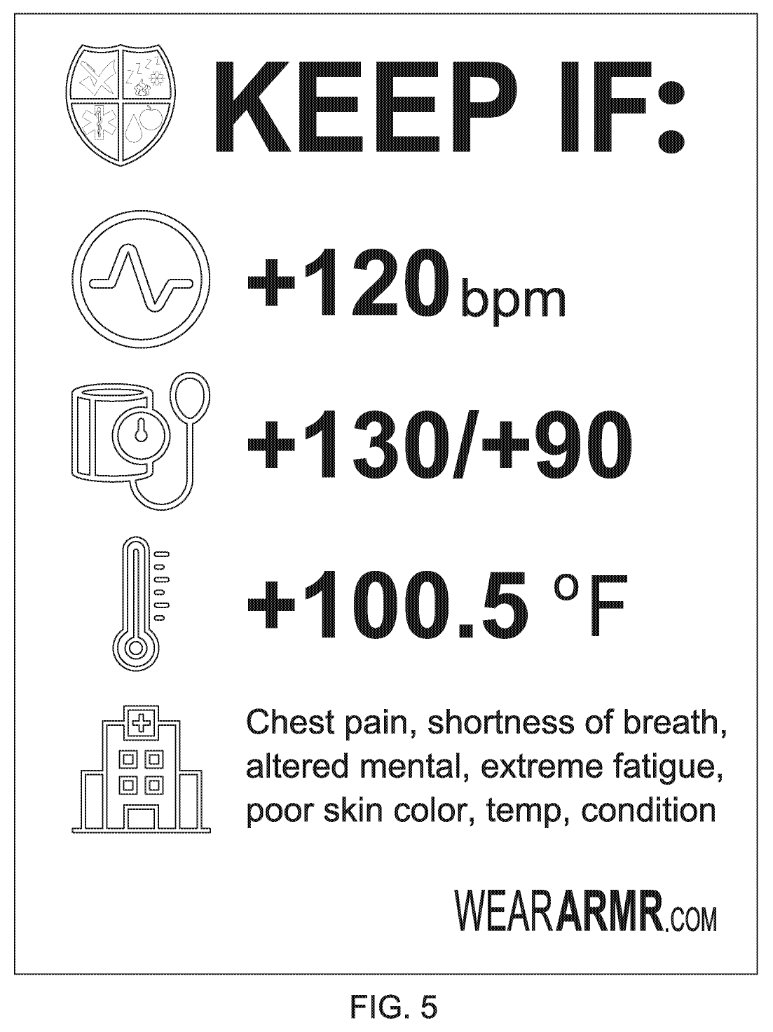

Referring to FIGS. 2 and 5, when a first responder is ready to exit the rehabilitation area, the Rehabilitation Manager completes the right side of the Manager Accountability Record. This includes documenting that the Individual Tracker has been reviewed, confirming that the Individual Tracker has been collected for storage or disposal in accordance with agency policies, recording the disposition of the responder (returned to duty or transported to hospital), recording the time of departure from the rehabilitation area, and initialing to confirm the checkout process.

If the first responder possesses an accountability tag, the Rehabilitation Manager retrieves the tag from the Accountability Tag Board and returns it to the responder before releasing them to return to duty. This ensures that accountability systems remain synchronized throughout the incident.

### Rehabilitation Area Placement and Layout

Referring to FIGS. 3 and 4, the Rehabilitation Placement recommendations provide guidance for establishing the location of the rehabilitation area on the emergency incident scene. Referring to FIG. 3, the Rehabilitation Map provides guidance for establishing the physical layout of the rehabilitation area. Referring to FIG. 4, the Rehabilitation Manager determines the location of the rehabilitation area, either as assigned by an Incident Commander or by utilizing the SUCK AIR acronym, which represents critical considerations for rehabilitation area placement: Safe location, Upwind and Uphill of hazards (smoke, chemical vapors, or other airborne contaminants), Close to incident, Kilometers (referring to distance: out of sight of the incident, but not too far away that personnel won’t walk), AIR (near where they have to go, such as an air truck to refill SCBA tanks, and with easy access to “open air” i.e. transportation to a hospital).

The rehabilitation area is organized into distinct sections, with each section typically accommodating between four and six first responders. Personnel retrieve the appropriate supplies indicated on their role cards from the equipment kit and set up the rehabilitation area using the Rehabilitation Map as a guide. This modular design allows for flexible scaling, with sections being added or removed as needed based on the number of first responders requiring rehabilitation and the available staffing. The layout is adaptable to different types of incidents, whether firefighting operations, hazardous materials incidents, search and rescue operations, or other emergency scenarios.

### Role-Specific Instruction Cards

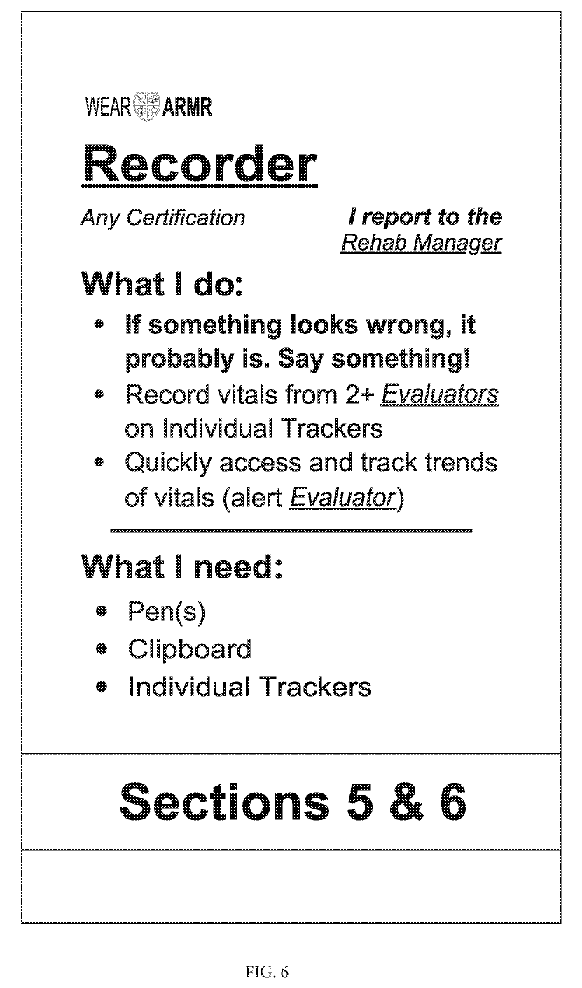

As depicted in FIGS. 6-11, each person staffing the rehabilitation area receives at least one role-specific instruction card designed to be worn on a lanyard around the neck. Each role card contains comprehensive written instructions including: the specific responsibilities of that role; the necessary equipment required to perform the role; any required certifications or qualifications; the supervisory relationships (i.e., to whom the role holder reports); and the designated location where the role is to be performed within the rehabilitation area. This design ensures that personnel can immediately understand and execute their assigned responsibilities without extensive prior training. As described below, one person may hold multiple roles.

The primary roles include: Rehabilitation Manager, who oversees the entire rehabilitation operation; Evaluators, who conduct medical screenings and assess vital signs; Recorders, who document medical information on Individual Tracker forms; and Transport Manager, who coordinates transport of first responders requiring higher-level medical care. Additional roles can be created as needed based on the scale of the incident and the number of responders requiring rehabilitation.

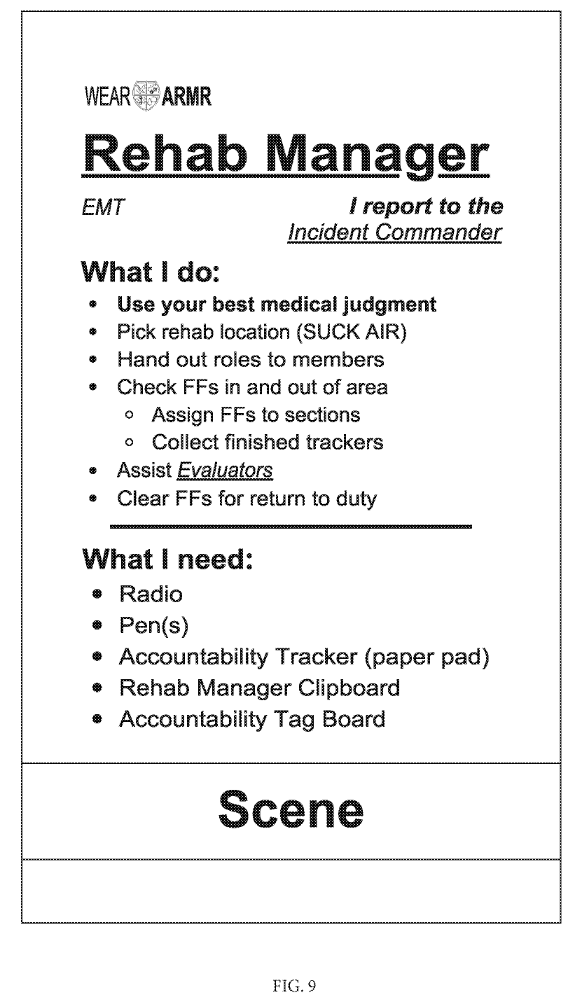

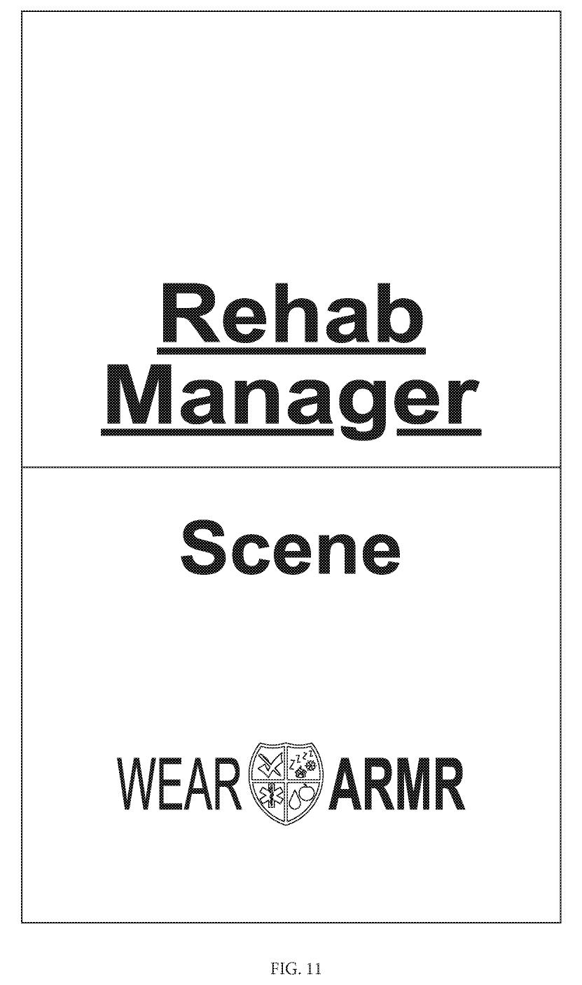

FIGS. 9 and 11 depict an exemplary Rehabilitation Manager role card. The Rehabilitation Manager is responsible for overseeing the entire rehabilitation operation, establishing the rehabilitation area location using the SUCK AIR considerations, coordinating with the Incident Commander, managing accountability tracking, and supervising all rehabilitation personnel. This role requires emergency medical technician certification or higher medical training and serves as the central point of coordination for the rehabilitation area.

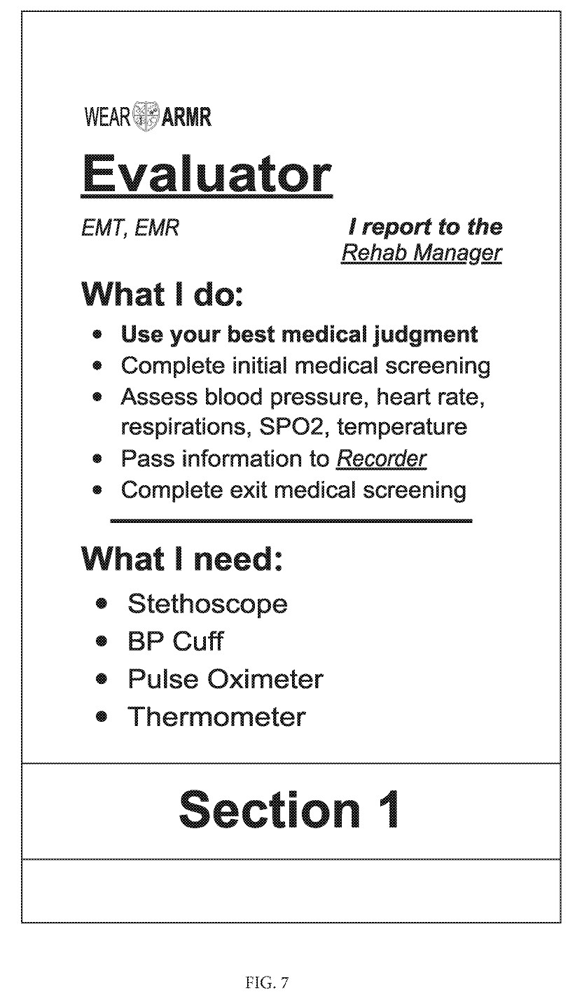

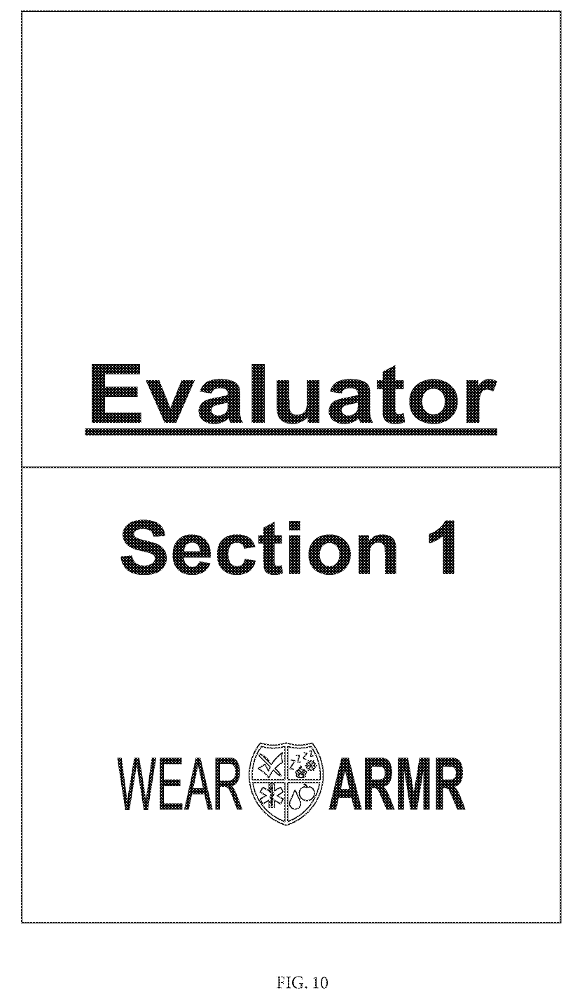

FIGS. 7 and 10 depict an exemplary Evaluator role card. Evaluators are responsible for conducting initial and exit medical screenings, assessing vital signs including heart rate, blood pressure, respiratory rate, and oxygen saturation, evaluating first responders for medical clearance to return to duty, and identifying individuals requiring transport to medical facilities. This role requires emergency medical technician certification or higher medical training and is positioned at designated evaluation stations within each rehabilitation section.

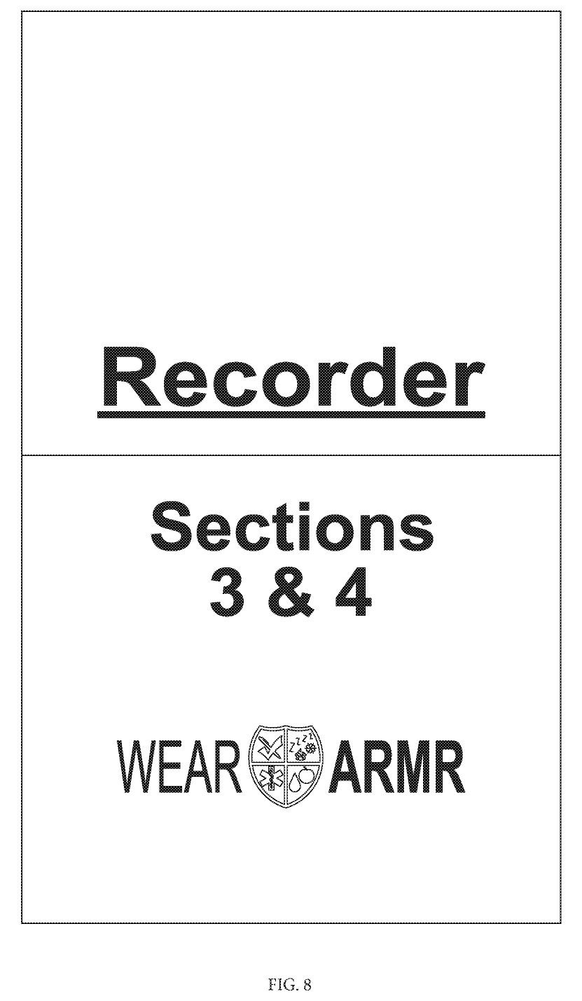

FIGS. 6 and 8 depict an exemplary Recorder role card. Recorders assist Evaluators by recording vital signs on Individual Tracker forms, tracking vital sign trends, managing medical supplies, ensuring availability of hydration and nutrition resources, and maintaining cleanliness of the rehabilitation sections. This role does not require medical certification but should include personnel with basic first aid training, and is positioned within each rehabilitation section to support the Evaluators.

The role cards depicted in FIGS. 6-11 represent exemplary configurations, and additional role cards may be created as needed based on the scale and complexity of the incident, available staffing, and specific agency requirements. The modular nature of the role card system allows for flexible adaptation to different incident types and operational environments while maintaining standardized procedures and accountability.

### Operational Workflow and Process Flow

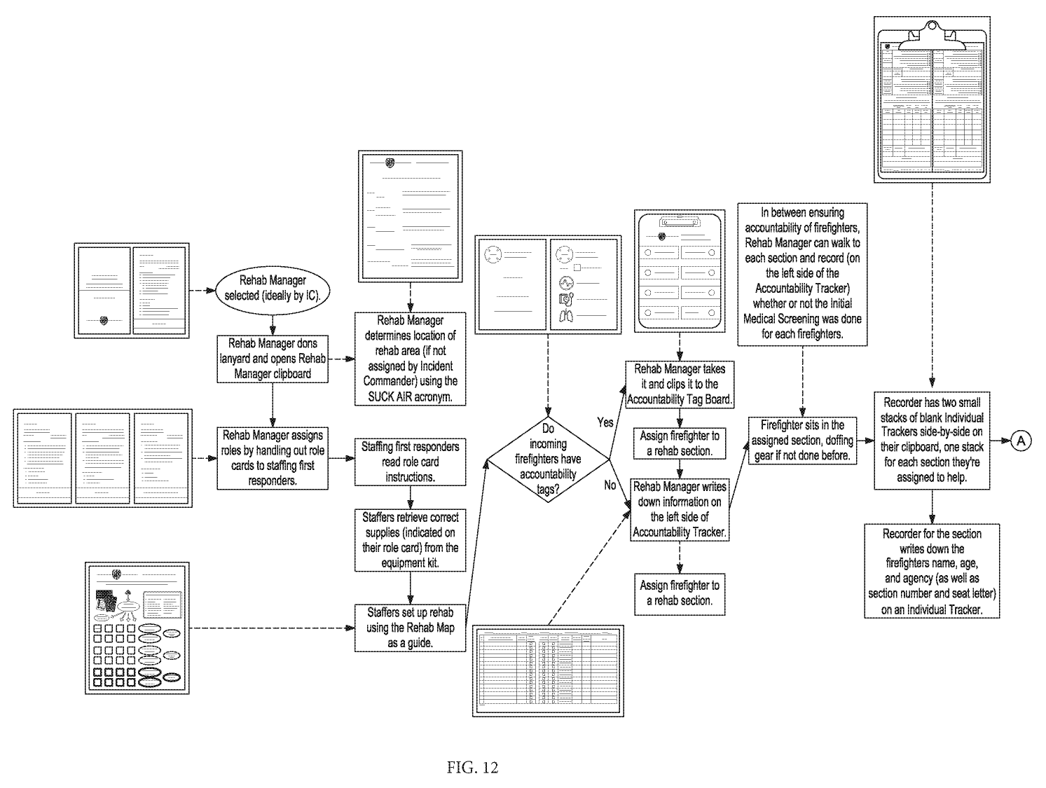

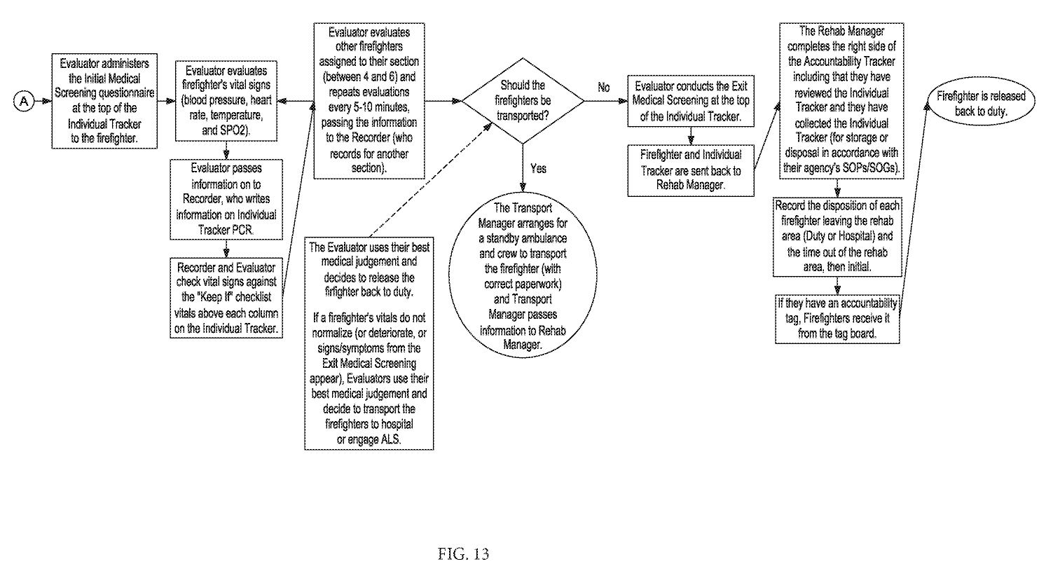

Referring now to FIGS. 12 and 13, these flowcharts illustrate the complete operational workflow of the emergency incident rehabilitation system, showing how the various components and roles interact to provide comprehensive rehabilitation services to first responders.

FIG. 12 depicts the initial setup and assignment process of the rehabilitation system. The process begins with selection of a Rehabilitation Manager, ideally by the Incident Commander. Once selected, the Rehabilitation Manager dons a lanyard and opens the Rehabilitation Manager clipboard. The Rehabilitation Manager then determines the location of the rehabilitation area, using the SUCK AIR acronym (if not already assigned by the Incident Commander) to ensure proper placement considering factors such as safety, uphill/upwind positioning, climate control, and accessibility.

The Rehabilitation Manager assigns roles to available personnel by distributing role-specific instruction cards to staffing first responders. Each card contains the necessary instructions for personnel to perform their assigned duties. The staffing first responders then read their role cards and follow the provided instructions to understand their responsibilities.

Following role assignment, staffers retrieve the correct supplies as indicated on their role cards and begin setting up the designated equipment kit. The rehabilitation area is established using the Rehabilitation Map as a guide, ensuring proper organization and layout of the various sections.

As firefighters begin arriving at the rehabilitation area, the system follows a decision point based on whether the firefighters possess accountability tags. If arriving firefighters have accountability tags, the Rehabilitation Manager collects and clips these tags to the Accountability Tag Board, then assigns the firefighters to a rehabilitation section. The Rehabilitation Manager completes the left side of the Accountability Tracker (with demograhic information) and can walk to each section and whether or not the Initial Medical Screening was completed for each firefighter.

If arriving firefighters do not have accountability tags, the Rehabilitation Manager writes down identifying information on the left side of the Accountability Tracker form, then assigns the firefighters to a rehabilitation section.

Once assigned to a section, firefighters sit in their designated section while donning appropriate gear if not completed previously. The Recorder for each section maintains two small stacks of blank Individual Trackers side by side on their clipboard, one stack for each section to which they are assigned to help. The Recorder then writes down each firefighter's name, age, and agency information, as well as the section number and seat letter, on separate Individual Tracker forms. This documentation ensures proper tracking and accountability throughout the rehabilitation process.

FIG. 13 depicts the medical evaluation, monitoring, and disposition process following the initial setup shown in FIG. 12. This flowchart continues from point A in FIG. 12 and illustrates the ongoing medical assessment and decision-making processes.

The process begins with the Evaluator administering the Initial Medical Screening questionnaire at the top of the Individual Tracker form to each firefighter. The Evaluator then evaluates each firefighter's vital signs, including but not limited to blood pressure, heart rate, temperature, and SpO2 (blood oxygen saturation). The Evaluator passes this information to the Recorder, who documents the vital signs on the Individual Tracker form. Notes or additional vital signs (such as SpCO, carbon monoxide saturation) are recorded on the blank reverse of the form.

The Evaluator continues this process for other firefighters assigned to their section, typically between four and six individuals. The Evaluator repeats vital sign measurements every five to ten minutes, communicating the updated information to the Recorder who maintains records for another section. The Recorder and Evaluator work together to check vital signs against the "Keep If" checklist appearing above each column on the Individual Tracker form to ensure measurements remain within acceptable parameters.

A critical decision point occurs when determining whether firefighters should be transported to a medical facility. If a firefighter's vitals deteriorate, do not normalize, or concerning signs and symptoms emerge from the Exit Medical Screening, the Evaluator exercises medical judgment to decide whether transport is necessary. The Evaluator uses their best medical judgment throughout this process, having the authority to decide at any point to release a firefighter back to duty, transport them to a hospital, or engage Advanced Life Support (ALS).

If transport is required, the Transport Manager arranges for a standby ambulance and crew to transport the firefighter. The Transport Manager completes the correct paperwork and passes all relevant information to the Rehabilitation Manager, who then relays this information to the appropriate personnel.

If the firefighter does not require transport, the Evaluator conducts an Exit Medical Screening at the top of the Individual Tracker form. Upon satisfactory completion, the firefighter and their completed Individual Tracker are sent back to the Rehabilitation Manager.

The Rehabilitation Manager then completes the right side of the Accountability Tracker, documenting that they have reviewed the Individual Tracker and collected it for storage or disposal according to their agency's standard operating procedures. The Rehabilitation Manager records the disposition of each firefighter leaving the rehabilitation area, noting whether they are returning to duty or being transported to a hospital, along with the time they exit the rehabilitation area and their initials.

If the firefighter possesses an accountability tag, they receive it back from the tag board upon checkout. The firefighter is then released back to duty, completing the rehabilitation cycle.

This systematic approach ensures continuous monitoring, proper documentation, and appropriate medical decision-making throughout the rehabilitation process, with multiple checkpoints to safeguard first responder health and safety.

### System Integration and Interrelationships

The various components of the system work together in an integrated manner. Each role's responsibilities, required equipment, required certifications, reporting relationships, and designated location are specified on the role card, ensuring that all personnel understand their functions within the overall system. This is why it is important that everyone receives at least one role card and reads and follows the instructions provided thereon.

The placement of the rehabilitation area on the incident scene and its internal layout are important for ensuring safety, proper rest, safe transport routes, and effective operations. The considerations for placement may vary depending on the type of incident. For firefighting operations, the area should be upwind of smoke; for hazardous materials incidents, the area should be upwind of chemical vapors and outside contamination zones; for search and rescue operations, the area should be accessible but not interfering with rescue operations.

Once first responders arrive at the rehabilitation area, the Rehabilitation Manager either collects accountability tags (attaching them to the Accountability Tag Board) or records information directly on the Manager Accountability Record. Responders are then seated in their assigned sections, where Evaluators conduct Initial Medical Screenings and assess vital signs, with Recorders documenting this information on Individual Tracker forms.

The evaluation process is repeated at regular intervals (approximately every five to ten minutes), with the Initial Medical Screening omitted on subsequent cycles. Both Evaluators and Recorders monitor for trends in vital signs and compare measurements against the "Keep If" checklist thresholds to identify responders requiring continued monitoring or intervention.

If an Evaluator determines, using medical judgment, that a first responder should be transported to a medical facility, the Transport Manager is immediately notified to arrange transportation. Conversely, if an Evaluator determines that a responder can return to duty, an Exit Medical Screening is conducted before the responder is sent to the Rehabilitation Manager for the checkout process.

### Operational Flexibility and Scalability

The system is designed to be highly flexible and adaptable to varying operational conditions and different types of emergency incidents. If a person staffing the rehabilitation area needs to be relieved, the lanyard and role card can be transferred to a replacement, thereby simplifying the hand-off process and ensuring continuity of operations. The new role holder can immediately reference the written instructions on the role card to understand their responsibilities.

The modular design allows the rehabilitation area to be scaled up or down as needed. Additional sections can be created when larger numbers of first responders require rehabilitation, while sections can be reduced or consolidated when demand decreases. This scalability is particularly valuable during extended operations, mass casualty incidents, or major disasters where large numbers of responders from multiple agencies may require rehabilitation services simultaneously.

If the rehabilitation area needs to be relocated, it can be moved incrementally, one section at a time, as responders complete their rest periods and depart. Alternatively, in emergency situations requiring immediate relocation (such as changing wind conditions during a hazardous materials incident or deteriorating structural conditions during a building collapse), the entire rehabilitation area can be moved quickly and re-established with minimal disruption to ongoing operations.

### Integration with Existing Accountability Systems

The system is designed to integrate seamlessly with existing accountability tag systems that may already be in use by fire departments, law enforcement agencies, emergency medical services, and other emergency response organizations. If responders already utilize an accountability tag system, that system can be incorporated into the rehabilitation area accountability protocols. Rehabilitation Managers collect tags as responders enter and return tags as responders depart, maintaining consistency with the overall incident accountability system.

When no accountability tag system exists, Rehabilitation Managers collect necessary accountability information directly from responders and record it on the Manager Accountability Record. In this manner, effective accountability is maintained regardless of whether a formal tag system is in place. In either case, the Rehabilitation Manager can seat responders immediately upon arrival and record detailed accountability information as time permits, ensuring that medical evaluation and rest begin without delay.

### Exercise of Medical Judgment

Throughout the rehabilitation process, Rehabilitation Managers and Evaluators exercise their medical judgment at all times. They may determine at any point that a first responder should be transported to a higher level of medical care, even if vital signs appear to be normalizing, if their clinical assessment indicates such action is warranted. Similarly, if an Evaluator determines that a responder can be safely released to return to duty, the appropriate steps outlined in the system must be followed, including conducting the Exit Medical Screening and completing the checkout process through the Rehabilitation Manager.

The system provides a standardized framework that supports, rather than replaces, clinical judgment. Medical personnel staffing the rehabilitation area apply their training and experience to make appropriate medical decisions for each individual responder, with the system ensuring that important steps are not overlooked and that consistent documentation is maintained.

### Critical Accountability and Evaluation Functions

The accountability and evaluation steps are of paramount importance to ensure that first responders are always locatable on an emergency incident scene and are efficiently and effectively evaluated for signs of medical distress or illness. This is equally important whether the responders are firefighters operating in a structure fire, law enforcement officers managing a prolonged standoff situation, emergency medical personnel treating patients during a mass casualty incident, or search and rescue teams working extended shifts during a disaster response.

All applicable role cards should be distributed to ensure that all necessary roles are fulfilled. In situations where fewer personnel are available than the number of distinct roles, individual persons may hold multiple roles simultaneously, provided they can effectively manage the combined responsibilities.

### Alternative Embodiments and Modifications

Various modifications and alternative embodiments can be implemented without departing from the scope of the invention. A tracking device and associated software could be incorporated to enable instant electronic check-in and check-out of responders entering and exiting the rehabilitation area. An electronic patient care record (PCR) system could be added to facilitate better tracking of vital signs, identification of trends over time, and recording of pertinent medical history information.

Standardized accountability tags, potentially incorporating quick response (QR) codes, radio frequency identification (RFID) chips, or similar machine-readable identifiers, could be implemented in combination with software systems to enhance the effectiveness and efficiency of the accountability processes. Such tags could enable rapid scanning for check-in and check-out, automated data transfer to electronic records, and real-time location tracking within the rehabilitation area.

The specific timing and methodology for completing documentation can be modified, provided that proper accountability and a high level of patient care are maintained. For example, roles may be combined such that an Evaluator simultaneously performs the Recording function, documenting vital signs and other medical information without requiring a separate Recorder. Individual Tracker forms could be physically attached to responders using an appropriate attachment system (such as wristbands or badge holders), allowing for immediate updating of medical information at the point of care.

The Rehabilitation Manager role could be combined with the Transport Manager role, with a single individual coordinating both the overall rehabilitation operations and the transportation of injured or ill responders to medical facilities. This consolidation may be appropriate for smaller incidents or when personnel resources are limited.

The Initial Medical Screening and Exit Medical Screening questionnaires can be customized to address specific concerns associated with different types of emergency incidents. For firefighting operations, questions may focus on smoke inhalation and heat exhaustion. For hazardous materials incidents, questions may address chemical exposure symptoms. For search and rescue operations, questions may emphasize signs of physical exhaustion and environmental exposure. The flexibility of the system allows for such customization while maintaining the overall structure and workflow.

In addition, there are additional role cards that could be utilized without departing from the spirit of the invention. For example, a Transport Manager Card could be included that describes how the Transport Manager, who reports to the Incident Commander, should use his or her best medical judgment to ensure:

proper accountability

at least one ambulance is always ready to transport injured responders; and

proper medical paperwork goes with each transported person.

The card could also describe how the Transport Manager would need a radio, pens and scratch paper, and a clipboard, by way of example.

### Applications Across Different Emergency Scenarios

The system is applicable to a wide variety of emergency scenarios involving different types of first responders. In firefighting operations, the system manages rehabilitation of firefighters experiencing heat stress, smoke inhalation, and physical exhaustion. In law enforcement operations, the system can be used to monitor officers during prolonged tactical operations, active shooter responses, or civil disturbance management.

During hazardous materials incidents, the system provides a structured approach to monitoring responders who may have been exposed to chemical, biological, or radiological hazards, ensuring that decontamination has been completed before entering the rehabilitation area and monitoring for delayed symptoms of exposure. In search and rescue operations, the system helps manage the rehabilitation of teams working extended shifts under physically demanding conditions.

During mass casualty incidents, the system can be employed to monitor the health of emergency medical personnel, firefighters, law enforcement officers, and other responders who are treating patients and managing the incident over extended periods. The system ensures that these responders receive adequate rest, hydration, and medical monitoring to prevent them from becoming casualties themselves.

In disaster response scenarios such as hurricanes, earthquakes, or floods, where responders from multiple agencies may work for days or weeks, the system provides a consistent rehabilitation framework that can be implemented by varying personnel as shifts change, ensuring continuity of care for responders throughout the duration of the response.

Furthermore, the organizational structure and documentation systems could be adapted for use in managing patients during mass casualty incidents. For example, the system could be employed as a framework for organizing and monitoring green-category (minor injury) or yellow-category (delayed treatment) patients during a mass casualty event, ensuring systematic evaluation and appropriate disposition of large numbers of patients with varying injury severities.

It should be understood, of course, that the foregoing relates to exemplary embodiments of the invention and that modifications may be made without departing from the spirit and scope of the invention as set forth in the following claims.

What is claimed is:

1. An emergency incident rehabilitation system for managing rehabilitation of first responders at emergency incident scenes, comprising:
a plurality of role-specific instruction cards, each card configured to be worn on a lanyard and comprising written instructions specifying role responsibilities, required equipment, required certifications, reporting relationships, and designated location for that role;
a rehabilitation area layout guide comprising instructions for establishing a rehabilitation area organized into distinct sections;
a manager accountability record form configured to track first responders entering and exiting the rehabilitation area;
a plurality of individual tracker forms, each form comprising fields for recording identifying information, initial medical screening results, vital signs measurements, and exit medical screening results; and
medical screening checklists comprising threshold values for vital signs.

2. The emergency incident rehabilitation system of claim 1, wherein the plurality of role-specific instruction cards comprises at least:
a rehabilitation manager card specifying oversight responsibilities;
at least one evaluator card specifying medical screening and vital signs assessment responsibilities; and
at least one recorder card specifying documentation responsibilities.

3. The emergency incident rehabilitation system of claim 1, further comprising an accountability tag board configured to hold accountability tags for first responders in the rehabilitation area.

4. The emergency incident rehabilitation system of claim 1, wherein each individual tracker form comprises fields for recording vital signs at multiple time intervals.

5. The emergency incident rehabilitation system of claim 1, wherein the vital signs measurements comprise blood pressure, heart rate, body temperature, and blood oxygen saturation.

6. The emergency incident rehabilitation system of claim 1, wherein the rehabilitation area layout guide comprises instructions for selecting a rehabilitation area location based on safety, proximity to the incident, accessibility, and environmental conditions.

7. The emergency incident rehabilitation system of claim 1, wherein the first responders comprise at least one of: firefighters, emergency medical services personnel, law enforcement officers, search and rescue team members, and hazardous materials specialists.

8. The emergency incident rehabilitation system of claim 1, wherein the medical screening checklists are adaptable to different types of emergency incidents.

9. A method for managing emergency incident rehabilitation for first responders at an emergency incident scene, comprising:
selecting a rehabilitation manager;
distributing role-specific instruction cards to personnel, wherein each card comprises written instructions specifying role responsibilities and designated location;
establishing a rehabilitation area organized into distinct sections according to a layout guide;
recording identifying information for first responders entering the rehabilitation area on a manager accountability record form;
conducting an initial medical screening for each first responder;
measuring vital signs for each first responder;
recording the vital signs measurements on an individual tracker form;
repeating the measuring and recording steps at regular intervals;
comparing the vital signs measurements to threshold values;
determining disposition for each first responder based on the vital signs measurements; and
recording departure information on the manager accountability record form for first responders exiting the rehabilitation area.

10. The method of claim 9, wherein the determining disposition step comprises either returning the first responder to duty or transporting the first responder to a medical facility.

11. The method of claim 9, further comprising conducting an exit medical screening prior to returning a first responder to duty.

12. The method of claim 9, further comprising collecting accountability tags from first responders upon entry and returning accountability tags to first responders upon exit.

13. The method of claim 9, wherein the regular intervals for repeating the measuring and recording steps comprise approximately five to ten minutes.

14. The method of claim 9, wherein the vital signs measurements comprise blood pressure, heart rate, body temperature, and blood oxygen saturation.

15. The method of claim 9, further comprising scaling the number of distinct sections in the rehabilitation area based on the number of first responders requiring rehabilitation.

16. The method of claim 9, further comprising relocating the rehabilitation area while maintaining accountability of first responders.

17. The method of claim 9, wherein the first responders comprise at least one of: firefighters, emergency medical services personnel, law enforcement officers, search and rescue team members, and hazardous materials specialists.

18. The method of claim 9, wherein the initial medical screening comprises questions adapted to the type of emergency incident.

19. A system for emergency incident rehabilitation comprising:
means for assigning roles to personnel comprising role-specific instruction cards worn on lanyards;
means for tracking first responders entering and exiting a rehabilitation area;
means for conducting medical screenings adapted to different types of emergency incidents;
means for recording medical evaluation data; and
means for determining whether to return first responders to duty or transport to a medical facility.

20. The system of claim 19, wherein the role-specific instruction cards comprise information enabling personnel to perform assigned roles with minimal prior training, and wherein the system is applicable to first responders comprising at least one of: firefighters, emergency medical services personnel, law enforcement officers, search and rescue team members, and hazardous materials specialists.
